(19) United States
(12) Patent Application Publication
(10) Pub. No.: US 2016/0324299 A1
(43) Pub. Date: Nov. 10, 2016

(54) TRANSFER DEVICE FOR MAKING UP KERATIN MATERIALS

(71) Applicant: L'OREAL, Paris (FR)

(72) Inventors: Henri Samain, Bievres (FR); Chrystele Gevrey, Sucy En Brie (FR); Franck Giron, Lagny sur Marne (FR)

(21) Appl. No.: 15/108,192

(22) PCT Filed: Dec. 19, 2014
(86) PCT No.: PCT/IB2014/067134

§ 371 Date: Jun. 24, 2016

(30) Foreign Application Priority Data
Dec. 27, 2013 (FR) 13 63630

(51) Int. Cl. A45D 40/30; A61K 8/49; B44C 1/17; A61K 8/02; A45D 40/26; A61K 8/34; A61Q 1/02

(52) U.S. Cl. CPC A45D 40/30; A61K 8/345; A61K 8/4913; A61Q 1/025; A61K 8/34; A61K 8/0204; A45D 40/261; B44C 1/1733

(57) ABSTRACT

The present invention relates to a transfer device for making up human keratin materials, including a substrate having at least one transfer surface, and a coat of at least one cosmetic colouring ink borne by the transfer surface and obtained by printing using at least one digital printer. The colouring ink being intended to be applied to the keratin materials so as to obtain by transfer a makeup result on the keratin materials. The colouring ink includes a dyestuff and a transfer compound other than the dyestuff and having a boiling point of greater than or equal to 120° C.

### BACKGROUND

It is difficult to make up keratin materials, especially the skin, by transferring a deposit of ink printed on a substrate. The reason for this is that the known inks are such that, once the printing is performed, they dry so quickly that transfer onto the keratin materials does not take place.

One way of solving this problem is to moisten the transfer surface or the area to be treated with a suitable solvent such as water. However, this method may be unacceptable due to the fact that it is not always possible to meter out precisely the amount of solvent to be applied, which may lead to “running” making the transfer onto the keratin materials irregular and/or imprecise and thus leading to an unsatisfactory makeup result.

Moreover, it is desirable for the makeup device to provide satisfactory transfer makeup in the case of a transfer performed immediately after printing, or within 30 minutes thereof, but also in the case of a transfer performed a few days or even a few months after printing.

In addition, it is also desirable for the pattern, once transferred onto keratin materials, especially the skin, to remain relatively stable. In other words, either immediately after transferring or, for example, within an hour of transferring, it is advantageous for the made-up area to be able to be touched, especially with the fingers, without deteriorating the pattern produced.

However, conventionally produced makeup coatings may not have satisfactory stability.

This lack of stability is not necessarily a problem, since high precision of the makeup pattern is not sought. On the other hand, in the case of precise patterns obtained by printing, it is important for the makeup obtained after transferring to be stable.

Finally, it may also be advantageous for the user to be able to retouch the transferred pattern within minutes of transferring, for example so as to be able to soften the contours and to smooth out the demarcations with the area not made up.

There is consequently a need for makeup devices that allow transfer makeup to be obtained by simple contact, without addition of solvent, whether the user seeks to transfer the pattern just after printing or after a longer or shorter period of storage of the device.

It would also be advantageous to obtain transfer makeup devices that allow the user, within minutes of transferring, to be able to retouch the pattern transferred onto the keratin materials, if necessary.

It is also sought to have available transfer makeup devices that make it possible to obtain a stable pattern within an hour of transferring.

The present invention is directed toward meeting all or some of these needs.

### SUMMARY

According to a first aspect, the present invention relates to a transfer device for making up human keratin materials, comprising:
a substrate having at least one transfer surface, and
a coat of at least one cosmetic colouring ink borne by the transfer surface and obtained by printing using at least one digital printer, the colouring ink being intended to be applied to, and especially placed in contact with, the keratin materials so as to obtain by transfer a makeup result on the keratin materials, the colouring ink comprising:
   a dyestuff, and
   a transfer compound other than the dyestuff and having a boiling point of greater than or equal to 120° C.

Unless otherwise mentioned, all the boiling points are measured at atmospheric pressure, i.e. 1013.25 mbar (corresponding to a pressure of 1013.25 hectopascals (hPa)).

The colouring ink may comprise water.

The presence in the colouring ink of a transfer compound of relatively high boiling point advantageously makes it possible to obtain good transfer of the colouring ink onto the keratin materials to be made up, during the application of the makeup device to the keratin materials.

In addition, the use of a coat of colouring ink obtained by printing using a digital printer advantageously makes it possible, when compared with standard makeup applications, to obtain a complex and customizable makeup result.

The dyestuff may comprise one or more dyes as described below.

Preferably, the colouring ink present on the device is not entirely dry 15 minutes after printing on the transfer surface, especially after a time of 24 hours and better still after a time of 7 days at 25° C. on contact with the air and at normal hygrometry (55% relative humidity).

The application of a colouring ink that is not entirely dry onto the keratin materials facilitates the transfer of the ink.

The colouring ink may be in liquid form when borne by the transfer surface and before application to the keratin materials.

Advantageously, the ink has a viscosity ranging from 1 mPa·s to 500 mPa·s and preferably from 1 mPa·s to 300 mPa·s at 25° C.

The viscosity of an ink of the invention may be measured via any process known to those skilled in the art, and especially according to the following conventional process. At 25° C. using a Rheomat 180 viscometer, equipped with a spindle rotating at 200 rpm, a person skilled in the art can select the spindle for measuring the viscosity from the spindles M1, M2, M3 and M4 on the basis of his general knowledge, so as to be able to perform the measurement.

In a particularly preferred manner, the colouring ink is capable of transferring onto the keratin materials without addition of an intermediary fluid compound, especially a liquid. In other words, the colouring ink may transfer onto the keratin materials by simple contact of the area intended to be made up with the said colouring ink, without it being necessary to apply an intermediary liquid intended to improve the transfer of the ink, as in the case of decal transfers.

The coat of colouring ink may comprise several inks.

The colouring inks deposited by printing may be deposited onto the transfer surface in the form of raster dots and/or of raster lines, so as to form a halftone image, for example a monochromatic or polychromatic image.

The coat of colouring ink may comprise several colouring inks of different colours, each deposited in raster dots.

The pattern formed by the colouring ink printed on the transfer surface may be of any type.

The pattern may reproduce the appearance of relief and/or colour heterogeneities of the skin, for example freckles or a mole.

The pattern formed by the coat of colouring ink borne by the transfer surface may be coloured when observed under white light in the visible region (400 nm-800 nm). As a variant, the pattern is colourless under white light in the visible region, but may appear coloured when submitted to a chemical and/or energy stimulus, such as exposure to UV (365 nm-400 nm), for example when the colouring ink contains a photochromic or fluorescent dyestuff.

Advantageously, the transfer compound(s) have a boiling point of greater than or equal to 120° C., in particular ranging from 120° C. to 350° C. and especially ranging from 120° C. to 300° C.

The transfer compound is advantageously in liquid form at room temperature (25° C.).

The transfer compound(s) are preferably chosen from: glycerol (boiling point: 290° C.), ethylene glycol (boiling point: 197° C.), diethylene glycol (boiling point: 245° C.), triethylene glycol (boiling point: 285° C.), 1,5-pentanediol (boiling point: 242° C.), 1-pentanol (boiling point: 138° C.), 1-hexanol (boiling point: 157° C.), benzyl alcohol (boiling point: 205° C.), 1-hexanal (boiling point: 130° C.), 1-heptanal (boiling point: 153° C.), 2-pyrrolidone (boiling point: 245° C.), N-methyl-2-pyrrolidone (boiling point: 203° C.), N-ethylpyrrolidine (boiling point: 21° C.), propylene carbonate (boiling point: 240° C.), 1,3-diaminopropane (boiling point: 140° C.), 2-imidazolidinone (boiling point: 131° C.), 2-amino-1-butanol (b.p.=178° C.), 2-aminopropanol (boiling point: 173° C.), ethanolamine (boiling point: 171° C.), butyl acetate (boiling point: 126° C.), and mixtures thereof.

According to a first embodiment of the invention, the transfer compound(s) are miscible in water at 25° C. (especially having a solubility in water of at least 5% by weight), and are preferably chosen from C5-C6 monoalcohols, C2-C6 polyols, C6-C10 esters, C5-C8 ketones (especially cyclic ketones), C6-C7 aldehydes, C3-C8 cyclic carbonates, C3-C8 cyclic ureas, C2-C6 amino alcohols, C3-C6 diamines, water-miscible amino silicones such as Silicone Quaternium-8 (INCI name) sold, for example, under the name Silsense Q-Plus Silicone by Noveon, PEG-7 amodimethicone (INCI name) sold, for example, under the name Silsense A-21 Silicone by Noveon, and mixtures thereof.

The colouring ink may comprise a plurality of different transfer compounds, preferably at least three different transfer compounds, preferably at least four different transfer compounds, the transfer compounds each having a boiling point of greater than or equal to 120° C., especially ranging from 120° C. to 350° C.

In one embodiment example, the transfer compounds comprise a mixture of at least two different C2-C6 polyols, especially of at least three different C2-C6 polyols and especially of at least four different C2-C6 polyols.

According to a second embodiment of the invention, the transfer compound(s) are immiscible in water (solubility in water at 25° C. of less than 5% by weight). Such transfer compounds may be chosen from the volatile or non-volatile oils usually used in cosmetics, which may be chosen from natural or synthetic carbon-based, hydrocarbon-based or fluoro oils, which are optionally branched, alone or as a mixture.

The term “non-volatile oil” means an oil that is capable of remaining on the skin at room temperature and atmospheric pressure for at least one hour, and especially having a non-zero vapour pressure at room temperature (25° C.) and atmospheric pressure of less than 0.01 mmHg (1.33 Pa).

Mention may be made in particular of non-volatile carbon-based oils, especially hydrocarbon-based oils of plant, mineral, animal or synthetic origin, such as liquid paraffin (or petroleum jelly), squalane, hydrogenated polyisobutene (Parleam oil), perhydrosqualene, macadamia oil, soybean oil, sweet almond oil, beauty-leaf oil, palm oil, grapeseed oil, sesame oil, corn oil, arara oil, rapeseed oil, sunflower oil, cotton oil, apricot oil, castor oil, avocado oil, jojoba oil, olive oil, cereal germ oil or shea butter oil; linear, branched or cyclic esters containing more than 6 carbon atoms, especially 6 to 30 carbon atoms, such as esters of lanolic acid, of oleic acid, of lauric acid or of stearic acid; esters derived from long-chain acids or alcohols (i.e. containing from 6 to 20 carbon atoms), especially the esters of formula RCOOR′ in which R represents a higher fatty acid residue comprising from 7 to 19 carbon atoms and R′ represents a hydrocarbon-based chain comprising from 3 to 20 carbon atoms, in particular C12-C36 esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, bis(2-ethylhexyl) succinate, diisostearyl malate, and glyceryl or diglyceryl triisostearate; higher fatty acids, especially of C14-C22, such as myristic acid, palmitic acid, stearic acid, behenic acid, oleic acid, linoleic acid, linolenic acid or isostearic acid; higher fatty alcohols, especially of C16-C22, such as cetanol, oleyl alcohol, linoleyl alcohol or linolenyl alcohol, isostearyl alcohol or octyldodecanol; and mixtures thereof.

Mention may also be made of decanol, dodecanol, octadecanol, benzyl alcohol, liquid fatty acid triglycerides of 4 to 10 carbon atoms such as heptanoic or octanoic acid triglycerides, caprylic/capric acid triglycerides; linear or branched hydrocarbons, of mineral or synthetic origin such as liquid paraffins and derivatives thereof, petroleum jelly, polydecenes and hydrogenated polyisobutene such as Parleam; synthetic esters and ethers especially of fatty acids, for instance purcellin oil, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate or fatty alkyl heptanoates, octanoates and decanoates; polyol esters such as propylene glycol dioctanoate, neopentyl glycol diheptanoate or diethylene glycol diisononanoate; and pentaerythritol esters; fatty alcohols containing from 12 to 26 carbon atoms, such as octyldodecanol, 2-butyloctanol, 2-hexyldecanol or 2-undecylpentadecanol.

Among the volatile compounds, mention may be made of non-silicone volatile oils, especially C8-C16 isoparaffins, such as isododecane, isodecane and isohexadecane.

More preferentially, mention may be made of alkanes that are liquid at room temperature, especially at 20° C., and more particularly decane, heptane, dodecane, isododecane, isohexadecane, cyclohexane and isodecane, and mixtures thereof.

Among the water-immiscible transfer compounds, use may be made of isododecane (boiling point: 180° C.), isopropyl myristate (boiling point: 168° C.), benzyl alcohol (boiling point: 205° C.), isostearyl alcohol (boiling point: 331° C.), isodecyl neopentanoate (boiling point: 272° C.), isononyl isononanoate (boiling point: 285° C.), oleyl alcohol (boiling point: 315° C.), 2-octyldodecanol (boiling point: 358° C.), isopropyl palmitate (boiling point: 340° C.), isopropyl isostearate (boiling point: 361° C.), and mixtures thereof.

The colouring ink according to the invention may also comprise waxes.

The term “wax” means a lipophilic compound, which is solid at room temperature (25° C.), with a reversible solid/liquid change of state, with a melting point of greater than or equal to 30° C., which may be up to 120° C. By bringing the wax to the liquid state (melting), it is possible to make it miscible with the oils that may be present and to form a microscopically homogeneous mixture, but on returning the temperature of the mixture to room temperature, recrystallization of the wax in the oils of the mixture is obtained. The melting point of the wax may be measured using a differential scanning calorimeter (DSC), for example the calorimeter sold under the name DSC 30 by the company Mettler.

The waxes may be hydrocarbon-based waxes, fluoro waxes and/or silicone waxes, and may be of plant, mineral, animal and/or synthetic origin. In particular, the waxes have a melting point of greater than 25° C. and better still greater than 45° C. As waxes that may be used in the colouring ink, mention may be made of beeswax, carnauba wax or candelilla wax, paraffin, microcrystalline waxes, ceresin or ozokerite; synthetic waxes such as polyethylene waxes or Fischer-Tropsch waxes, silicone waxes such as alkyl or alkoxy dimethicones containing from 16 to 45 carbon atoms.

The nature and amount of the waxes depend on the desired mechanical properties and textures. As a guide, the colouring ink may contain from 0.01% to 30% by weight and better still from 1% to 20% by weight of waxes relative to the total weight of the colouring ink.

The colouring ink advantageously comprises a mixture of a plurality of transfer compounds, each being miscible in water at 25° C. As a variant, the colouring ink comprises a mixture of a plurality of transfer compounds, each being water-immiscible. As another variant, the colouring ink comprises a mixture of a plurality of transfer compounds and comprises both one or more water-miscible transfer compounds and one or more water-immiscible transfer compounds.

In one embodiment example, the colouring ink also comprises an organic solvent that is liquid (at 25° C.) with a boiling point of less than 120° C. As examples of such organic solvents, mention may be made of n-hexane (boiling point: 69° C.), cyclohexane (boiling point: 81° C.), ethyl acetate (boiling point: 76° C.), n-heptane (boiling point: 98° C.), isobutyl acetate (boiling point: 116° C.), methyl acetate (boiling point: 57° C.), ethanol (boiling point: 78° C.), butanol (boiling point: 117° C.), isopropanol (boiling point: 81° C.), n-propanol (boiling point: 97° C.), and mixtures thereof.

The colouring ink may be in the form of an aqueous solution, an aqueous gel or an emulsion.

The transfer compound(s) may be present in the colouring ink in a mass content ranging from 2% to 80%, preferably ranging from 10 to 70%, preferably ranging from 15% to 70% and particularly preferably ranging from 20% to 60%, relative to the total mass of the ink.

The use of a sufficient amount of transfer compounds in the colouring ink makes it possible to further improve its transfer onto the keratin materials, and in particular to dispense with the use of an intermediary fluid compound intended to improve the transfer.

Preferably, the colouring ink does not comprise any pigment.

Preferably also, the colouring ink does not comprise any particulate filler.

The dyestuff may be present in the colouring ink in a mass content ranging from 0.01% to 60%, preferably ranging from 0.1% to 40% and preferentially ranging from 0.1% to 20% relative to the total mass of the ink.

The colouring ink may comprise water in a mass content ranging from 19.9% to 97.9% by weight, preferably ranging from 29.9% to 89.9% by weight and preferentially ranging from 39.9% to 79.9% relative to the total mass of the ink.

Substrate and Transfer Surface

In one embodiment example, the substrate used in the invention comprises at least one translucent or transparent area.

The translucent or transparent area allows a user to see through the substrate and thus to visualize more easily the surface to be made up and/or treated before transferring the cosmetic ink. The presence of a translucent or transparent area thus advantageously contributes towards facilitating the production of a precise makeup result on the keratin materials.

The translucent or transparent area of the substrate can be totally or partly superposed with the coat of cosmetic ink, and especially may overlap with it.

The coat of cosmetic ink may be superposed in its entirety on the translucent or transparent area of the substrate. As a variant, only part of the coat of cosmetic ink is superposed on the transparent area of the substrate.

The substrate may be made of a transparent or translucent material. In this case, the translucent or transparent area extends over the entire surface of the substrate.

As a variant, the substrate is opaque over all or part of its surface.

The substrate may comprise a material in sheet form, especially a transparent material.

The substrate may be a flexible sheet or a rigid plate. It may be made of plastic (for example polyethylene or polystyrene). It may be woven or nonwoven. It may be made of organic or mineral material. It may be an aluminium foil.

The substrate is preferentially based on a non-absorbent material, for example a plastic film. The substrate is advantageously non-porous, at least on the face intended to receive the print.

The transfer surface may retain the cosmetic ink by capillary action.

The transfer surface may or may not be flat.

The transfer surface of the substrate may be defined by all or part of: the outer surface of an applicator roller, the surface of an applicator pad, an element in sheet form, a patch, the surface of a porous foam, especially a sponge or a wipe, a coarse brush, a fine brush or a flocked tip.

The applicator roller may have the form of a right cylinder. In one variant, the roller has the form of an irregular cylinder, for example the form of an hourglass.

In one variant, the roller is “premoulded”, i.e. it has an initial non-flat form corresponding to the general form of the area to be made up, for example the negative of the lips, of an eye socket, of an ankle or of a forearm.

In one variant, the substrate is plated at the time of transfer against an imprint of the area to be made up, so that the transfer surface reproduces the relief of the area to be made up.

The transfer surface is defined, for example, by all or part of the surface of a deformable sheet mounted on the surface of an applicator roller or a pad.

The transfer surface may be elastically deformable. Thus, in a first configuration, the transfer surface may be flat, and, in a second configuration, the transfer surface may be incurved.

In one variant, the substrate is configured so that the transfer surface takes a first form, for example substantially flat, during printing, and a second form, different from the first, during the application of the colouring ink to the keratin materials. The second form advantageously corresponds to the form of the surface of the keratin materials intended to be coated with the colouring ink, for example the form of the nails or of a part of the face.

The substrate is preferentially based on a non-absorbent material, for example a plastic film. The substrate is advantageously non-porous, at least on the face intended to receive the print.

In one embodiment, when the colouring ink is intended to be applied to the cheeks and/or the nails, the substrate may have a thickness of greater than or equal to 1 mm, especially 3 mm, for example ranging from 1 to 5 mm.

In one embodiment, when the colouring ink is intended to be applied to the area around the eyes and/or to the lips, the substrate may have a thickness of greater than or equal to 3 mm, especially 1 mm, for example ranging from 3 to 20 mm.

In one embodiment example, when the colouring ink is intended to be applied to the nose and/or in the area of the ears, the substrate may have a thickness of greater than or equal to 1 cm, especially 3 cm, for example ranging from 1 to 4 cm.

Thus, the substrate advantageously has a thickness adapted to the area of keratin materials to be made up.

The thickness of the substrate corresponds to its maximum dimension measured perpendicular to the transfer surface.

The substrate may have a variable thickness.

The substrate may be premoulded.

In one embodiment example, the substrate comprises an indication printed or not with the same ink as that intended to be transferred. The indication states, for example, the nature of the keratin materials intended to be made up with the colouring ink or illustrates to scale, enlarged, reduced or otherwise and “right-side up” the pattern deposited “wrong-side up” on the substrate.

In one embodiment example, the transfer surface is detachable from a part of the substrate.

The substrate may be reusable.

For example, printing is performed on the substrate, which is accessible for the transfer, but does not leave the printer. Thus, after use, the printer can reintegrate the substrate, clean it and make it ready for a new print.

The substrate may comprise, before printing, a coating which may be coloured and which may itself be capable of transferring on to human keratin materials. The coating may be coloured a colour that is difficult to print, for example white or black, or a colour close to the flesh tone of the skin to be made up, such as a flesh colour. As a variant, the coating is not itself coloured.

This or these coatings may also be applied to the substrate after it has been printed with the colouring ink.

According to another of its aspects, the present invention relates to a cosmetic assembly comprising, in the same packaging, a plurality of devices according to the invention, the devices differing by the chemical nature of the colouring ink they bear and/or by the pattern thereby formed and/or by the form of the transfer surface intended to engage with the keratin materials.

A subject of the present invention is also a process for manufacturing a device as defined above, comprising the step that consists in printing, using at least one digital printer, on a transfer surface at least one cosmetic colouring ink, the ink comprising:
a dyestuff, and
a transfer compound other than the dyestuff, having a boiling point of greater than or equal to 120° C.

In one embodiment example, the coat of colouring ink is printed in a predefined pattern. The process may comprise a step of choosing and/or making the pattern by a user and of transmitting, by means of a machine connected to at least one printer that performs the printing, information relating to this pattern.

The machine may be a computer, an advanced portable telephone, also known as a “smartphone”, or a tablet computer. The machine may be connected physically and/or by means of a data exchange network to the said printer.

The printer may be an inkjet printer, for example a thermal or piezoelectric printer, a sublimation printer or a 3D printer. The printer may be a standard commercial printer modified so as to be used in the context of the invention.

In one embodiment example, the printing is performed directly onto a non-flat transfer surface, corresponding especially to the outer surface of a roller.

The printing may use several different inks according to the invention, especially inks of different colours.

The printing may use at least three, especially at least four, five, six, seven, eight, nine, ten, eleven or twelve colouring inks of different colours.

The printing may use only colouring inks of primary colours. As a variant, the printing uses both colouring inks of primary colours and at least one colouring ink of non-primary colour.

The printing of the coat of colouring ink may be three-colour or four-colour printing.

The pattern obtained by printing may comprise several areas of different colours. As a variant, the pattern obtained by printing is a flat tint.

The coat of colouring ink may be deposited in several printing passes. In other words, a first fraction of the colouring ink may first be printed onto the transfer surface, followed by a second fraction of the colouring ink on all or part of the first fraction.

In one embodiment example, the substrate is coated with a coloured coating in dry form, the coating comprising a pigment and/or a dye, the colouring ink being printed onto the coating.

The printing may also follow geometrical rectification rules. Insofar as the transfer surface is deformable, during the application, the pattern may be geometrically deformed (for example extension in one of the two dimensions). As a result, the pattern is printed with a geometrical deformation (in the present case reduction according to the deformable dimension(s)) such that, after application, the pattern is at the desired scale. Geometrical rules: either universal or specific, may be applied to the pattern to be printed on the transfer surface so that the pattern has the desired form after transfer onto the area of the keratin materials to be treated. The use of such rectification rules is particularly advantageous with a substrate that has a transfer surface bearing reliefs, in particular in order to embrace the form of an imprint, as will be seen later. Use may be made in particular of specific geometrical rules adapted to the area to be treated and/or to the desired pattern.

The present invention also relates to a process for making up human keratin materials, comprising the step consisting in applying to the keratin materials at least one colouring ink present on a device according to the invention, the colouring ink especially being applied to a skin surface, for example the eyelids, the nails, the lips or the scalp, or to the hair.

Advantageously, the colouring ink is not entirely dry on the substrate when it is applied to the keratin materials. The colouring ink may be in fluid form when it is applied to the keratin materials, especially when the transfer compound(s) are fluid at a temperature of 20° C.

All or part of the coat of ink borne by the transfer surface may be applied by transfer to the keratin materials.

In one embodiment example, at least 25% by mass, especially 50%, especially 75% and especially substantially all of the coat of colouring ink initially present on the transfer surface is preferably applied by transfer to the keratin materials, preferably without addition of an intermediary fluid compound.

In one embodiment example, the application of the colouring ink is performed by application with pressure of the transfer surface onto the keratin materials, the pressure especially being applied for a time of 1 to 10 seconds.

The application of the colouring ink to the surface to be treated may be performed without rubbing.

In one implementation example according to the invention, the process also comprises a step of finishing the makeup obtained on the keratin materials, for example so as to attenuate the demarcations between a made-up area and an area not made up. The finishing of the makeup obtained may comprise a step of spreading the colouring ink to produce shading-off, for example.

The user may perform finishing before and/or after the transfer of the colouring ink onto the keratin materials.

In one embodiment example, the process thus comprises a step of finishing the pattern formed with the colouring ink borne by the transfer surface and/or a step of finishing the makeup obtained on the keratin materials, so as to attenuate the demarcations between a made-up area and an area not made up, the finishing being performed, for example, by exerting friction on only a part of the transferred pattern, for example on its upper part in the case of a pattern applied to the eyelid.

Advantageously, the area of keratin materials intended to receive the colouring ink has not been pretreated at the time of application of the colouring ink.

In one embodiment example, the keratin materials intended to be coated with the colouring ink have not been covered, before application of the colouring ink, with an intermediary fluid compound intended to improve the transfer of the colouring ink and/or the process lacks a step of addition to the colouring ink borne by the transfer surface of an intermediary fluid compound intended to improve the transfer.

As a variant, the area of keratin materials intended to be coated with the colouring ink has been covered, before application of the colouring ink, with an intermediary fluid compound for improving the transfer of the colouring ink and/or an intermediary fluid compound intended to improve the transfer has been added to the colouring ink borne by the transfer surface before its application to the keratin materials.

As another variant, in particular if the transfer compound(s) are not fluid at a temperature of 20° C., an intermediary fluid compound, which is especially different from the transfer compound(s), is added to the transfer compound(s) present in the coat of colouring ink, the intermediary compound making it possible to fluidize, optionally after evaporation of a solvent, the said transfer compound(s), the said intermediary compound having, for example, a boiling point of greater than or equal to 120° C.

The intermediary compound, for instance ethanol or isododecane, for improving the transfer of the colouring ink is added to the colouring ink, especially while the said ink is in contact with the area of the keratin materials onto which it is intended to transfer.

In one embodiment example, the addition of the intermediary fluid compound for improving the transfer of the colouring ink makes it possible only to dissolve all or part of the colouring ink and not, for example, the substrate of the makeup device and/or a layer of adhesive.

When an intermediary compound that is useful for fluidizing the transfer compound(s) is used, the intermediary compound may be added within the colouring ink to the transfer compound(s) after printing.

The intermediary compound may be added, within the colouring ink, to the transfer compound(s) by any known means, especially by spraying.

The intermediary compound is preferably added to the transfer compound(s) before the application of the colouring ink to the keratin materials, while the colouring ink is still borne by the transfer surface.

The transfer compound(s) may not be fluid at a temperature of 20° C., whereas the colouring ink borne by the transfer surface is fluid at a temperature of between 30° C. and 60° C., for example between 30° C. and 50° C.

In one embodiment example, the colouring ink is brought to a temperature of between 30° C. and 60° C. before being applied to the keratin materials. As a variant, the colouring ink is brought to a temperature of between 30° C. and 60° C. while it is in contact with the area of the keratin materials intended to be coated with the colouring ink.

When the colouring ink is intended to be brought to a temperature of between 30° C. and 60° C. prior to its application, the colouring ink brought to this temperature may be applied to the nails so as to produce a makeup result thereon by transfer. The colouring ink may be brought to a temperature of 30-60° C., for example by means of a hair dryer or of an infrared source.

When the transfer surface is detachable from a part of the substrate, the user can first detach the transfer surface from the rest of the substrate and then apply by transfer onto the keratin materials the colouring ink present on the transfer surface thus detached.

The colouring ink may be brought to a temperature of between 30° C. and 60° C. by being placed close to a heating member. As a variant, the colouring ink obtained just after printing may already be at such a temperature; the user can then apply to the keratin materials the colouring ink at this temperature, before it cools.

According to yet another aspect, the present invention relates to a cosmetic assembly for manufacturing a device according to the invention, comprising, in the same packaging:
a) a printer cartridge comprising a cosmetic colouring ink, the colouring ink comprising:
   a dyestuff, and
   a transfer compound other than the dyestuff and having a boiling point of greater than or equal to 120° C., and
b) a transfer surface intended to be printed with the colouring ink.

Measurement of the Ability of the Colouring Ink to Transfer without the Need to Add an Intermediary Fluid Compound

As mentioned above, the colouring ink is, particularly preferably, capable of transferring onto the keratin materials without the addition of an intermediary fluid compound.

To check whether a given colouring ink has this property, the coat of colouring ink under consideration borne by a surface is placed in contact with a sample of artificial skin sold by the company Beaulax under the brand name Bioskin ref #white 061031-2.

The contact is performed for a time of 1 s by applying a pressure of 5000 pascals (i.e. 50 g/cm²) under atmospheric temperature and pressure conditions (20° C. and 1 bar). No intermediary fluid compound is added either to the colouring ink or to the sample before or during the contact.

A visual evaluation is performed.

If the colouring ink transfers onto the keratin materials, then the colouring ink is considered as being capable of transferring onto the keratin materials without the addition of an intermediary fluid compound.

Dyestuff

The colouring ink may comprise one or more dyestuffs chosen from water-soluble dyes, liposoluble dyes, pulverulent dyestuffs such as pigments, especially nacres, and glitter flakes, or alternatively colouring polymers.

The dyestuff(s) may be present in the colouring ink in a content ranging from 0.01% to 40% by weight, preferably from 0.1% to 30% by weight and preferentially ranging from 0.5% to 20% by weight relative to the total weight of the colouring ink.

The term “pigments” should be understood as meaning white or coloured, mineral or organic particles of any form, which are insoluble in the cosmetic medium, and which are intended to colour the colouring ink.

The term “nacres” should be understood as meaning iridescent particles of any shape, in particular produced by certain molluscs in their shell, or else synthesized.

The pigments may be white or coloured, and mineral and/or organic. Among the mineral pigments that may be mentioned are titanium dioxide, optionally surface-treated, zirconium oxide or cerium oxide, and also zinc oxides, iron (black, yellow or red) oxides or chromium oxides, manganese violet, ultramarine blue, chromium hydrate and ferric blue, and metal powders, for instance aluminium powder and copper powder.

Among the organic pigments that may be mentioned are carbon black, pigments of D&C and FD&C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment and also nacreous pigments based on bismuth oxychloride.

Among the water-soluble dyes, mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll and methylene blue. Among the liposoluble dyes, mention may be made of Sudan Red III (CTFA: D&C Red 17), lutein, quinizarine green (CTFA: D&C Green 6), alizurol purple SS (CTFA: D&C Violet 2), Sudan brown, D&C yellow 11, D&C Orange 5, quinoline yellow, curcumin, carotenoid derivatives such as lycopene, beta-carotene, bixin or capsanthin, and mixtures thereof. The dyeing polymers are generally copolymers based on at least two different monomers, at least one of which is a monomeric organic dye. Such polymeric dyes are known to those skilled in the art. Reference may be made, for example, to the following documents: U.S. Pat. No. 5,032,670; U.S. Pat. No. 4,999,418; U.S. Pat. No. 5,106,942; U.S. Pat. No. 5,030,708; U.S. Pat. No. 5,102,980; U.S. Pat. No. 5,043,376; U.S. Pat. No. 5,104,913; U.S. Pat. No. 5,281,659; U.S. Pat. No. 5,194,463; U.S. Pat. No. 4,804,719; WO 92/07913 or EP 1 048 282.

The colouring ink may comprise one or more dyestuffs, especially photochromic pigments, i.e. dyestuffs which have the property of changing colour when they are irradiated with a light source of a certain frequency, and then of regaining their initial colour, or a similar colour, when the irradiation is stopped. Among the photochromic dyestuffs, mention may be made especially of:
complex mineral photochromic compounds and more particularly doped aluminosilicates and metal oxides and metal oxide hydrates, such as those described in WO-A-02/36083;
photochromic naphthopyran compounds, especially 3H-naphtho[2,1-b]pyrans or 2H-naphtho[1,2-b]pyrans, for instance 3,3-bis(4-methoxyphenyl)-6-morpholino-3H-naphtho[2,1-b]pyran, 3-phenyl-3-(4-morpholinophenyl)-6-morpholino-3H-naphtho[2,1-b]pyran, 3-phenyl-3-(4-piperidinophenyl)-6-morpholino-3H-naphtho[2,1-b]pyran, 3-phenyl-3-(4-piperidinophenyl)-6-carboxymethyl-9-N-dimethyl-3H-naphtho[2,1-b]pyran or 2-phenyl-2-(4-piperidinophenyl)-5-carboxymethyl-9-N-dimethyl-2H-naphtho[1,2-b]pyran. Such compounds are described in patent application EP-A-1 410 785;
diarylethene or fulgide compounds such as those described in patent application EP-A-938 887.

The colouring ink may also comprise one or more fillers, especially in a content ranging from 0.01% to 50% by weight, relative to the total weight of the colouring ink, preferably ranging from 0.010% to 30% by weight.

The term “fillers” should be understood as meaning colourless or white, mineral or synthetic particles of any shape, which are insoluble in the medium of the colouring ink, irrespective of the temperature at which this ink is manufactured.

These fillers serve especially to modify the rheology or texture of the colouring ink.

The fillers may be mineral or organic and of any shape, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powder (Orgasol® from Atochem), poly-β-alanine powder and polyethylene powder, tetrafluoroethylene polymer (Teflon®) powder, lauroyllysine, starch, boron nitride, hollow polymer microspheres such as polyvinylidene chloride/acrylonitrile microspheres, for instance Expancel® (Nobel Industrie), acrylic acid copolymer microspheres (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

The colouring ink may also comprise an additional polymer such as a film-forming polymer. The term “film-forming polymer” means a polymer that is capable of forming, by itself or in the presence of an auxiliary film-forming agent, a continuous film that adheres to a support, especially to keratin materials. Among the film-forming polymers that may be used in the colouring ink, mention may be made of synthetic polymers, of radical type or of polycondensate type, polymers of natural origin and mixtures thereof, in particular acrylic polymers, polyurethanes, polyesters, polyamides, polyureas, and cellulose-based polymers, for instance nitrocellulose.

Needless to say, a person skilled in the art will take care to select this or these optional additional compound(s), and/or the amount thereof, such that the advantageous properties of the colouring ink are not, or are not substantially, adversely affected by the envisaged addition.

Cosmetically Acceptable Medium

The colouring ink according to the invention constitutes a cosmetically acceptable medium, i.e. a medium that is compatible with keratin materials such as the skin of the face or the body, the lips, the hair, the eyelashes, the eyebrows and the nails.

### DESCRIPTION OF THE FIGURES

The invention may be understood more clearly on reading the following description of non-limiting implementation examples thereof, and on examining the attached drawing, in which:

FIG. 1 shows an example of a makeup device according to the invention,

FIG. 2 is a section along II-II of the makeup device of FIG. 1,

FIGS. 3 to 5 represent different steps of an example of a makeup process according to the invention,

FIGS. 6 and 7 represent examples of cosmetic assemblies according to the invention,

FIG. 8 is a block diagram illustrating various steps for manufacturing a makeup device according to the invention,

FIGS. 9 to 11 illustrate variants of the makeup process according to the invention, and

FIG. 12 shows an example of a transfer makeup result obtained using a makeup device according to the invention.

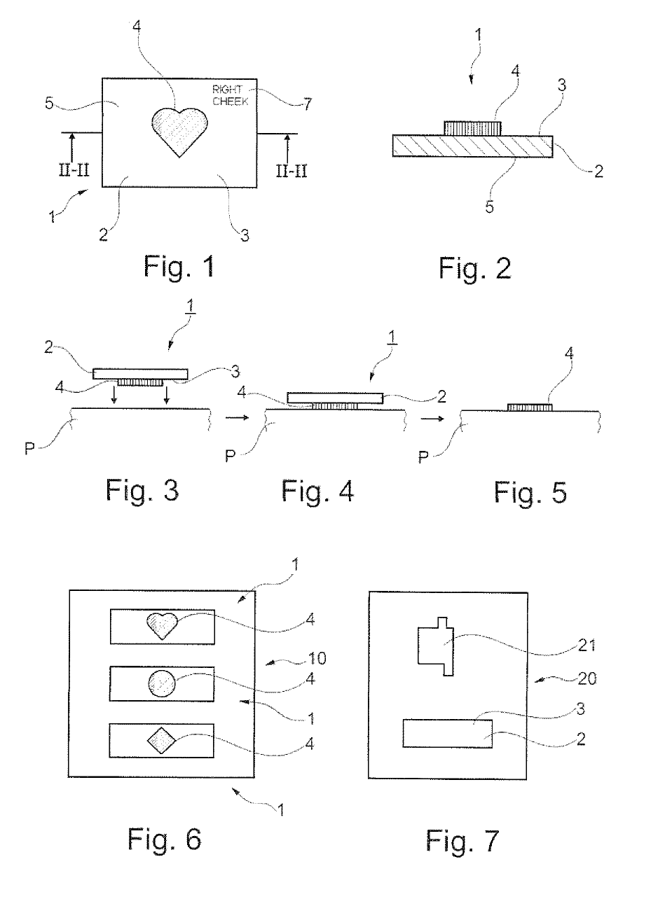

FIGS. 1 and 2 show a makeup device 1 according to the invention, comprising a substrate 2 whose front side defines a transfer surface 3. The device 1 may, as illustrated, have only one face defining the transfer surface 3, bearing a coat 4 of at least one cosmetic colouring ink according to the invention.

In one variant, not shown, two transfer surfaces 3 are defined by the two opposite faces of the substrate 2. In this case, these surfaces may bear coats of different cosmetic colouring inks, these coats possibly differing by their colour, the nature of the colouring inks borne and/or by the patterns formed.

In the device 1 illustrated in FIGS. 1 and 2, the coat of colouring ink 4 borne by the transfer surface 3 was deposited by printing using a digital printer, which deposits the ink dots in correspondence with the pixels of an image to be reproduced, for example in the form of rasters. This coat 4 is not entirely dry at the time of application to the keratin materials and comprises, for example, a transfer compound that is fluid at a temperature of 20° C.

Each colouring ink comprises a dyestuff and a transfer compound different from the dyestuff and having a boiling point of greater than or equal to 120° C. Each colouring ink may comprise a plurality of different transfer compounds, as detailed previously.

The coat 4 may form any type of pattern, for example in the form of a heart as illustrated.

The substrate 2 may have at least one non-opaque area 5, which is transparent or translucent, and which may totally or partly be superposed with the coat 4. The transparent area 5 allows the user to see through the substrate 2 and thus to visualize the surface to be made up through the device 1 when this device is superposed on the said surface.

All of the coat 4 may, as illustrated, be superposed on the transparent area 5. In one variant, not shown, only part of the coat 4 is superposed on the transparent area 5.

The substrate 2 may be made of a transparent material. The transparent area 5 then extends over the entire surface of the substrate 2.

The substrate 2 may bear an indication 7, for example print, which gives information regarding a recommended positioning for the makeup, for example “right cheek” as illustrated, or a reproduction of the place and scale, reduced or not, of the pattern to be transferred or the nature of the keratin materials intended to be made up with the colouring ink, or the like, and may also provide information regarding the colour and/or pattern reference.

The substrate 2 is preferably made of a flexible material. As a variant, the substrate 2 is made of a rigid or semi-rigid material.

All or part of the area of the transfer surface 3 superposed on the coat 4 is preferably smooth and has a roughness of less than or equal to 1 mm, especially between 1 and 100 μm and preferably less than or equal to 50 μm. The roughness is measured using a roughness meter, the tip of which has a radius of curvature of 10 mm, and the force of which, applied to the material to be characterized, is 6 mN.

FIGS. 3 to 5 schematically show various steps of an example of a makeup process according to the invention. As illustrated, the device 1 is first brought close to the area of skin P to be made up, which is preferably dry, so as to place the coat 4 in contact with the area of skin P to be made up, and the user then applies a pressure allowing the colouring ink to be transferred onto the area of skin P to be made up. During the contact with the keratin materials, the substrate 2 is preferably not moved sideways so as not to affect the appearance of the transferred pattern.

The pattern transferred onto the keratin materials corresponds to the pattern formed by the coat 4 when it is present on the substrate 2 (i.e. when it has not yet been transferred onto the keratin materials to be made up).

In one example, not shown, the process also comprises a step of finishing the makeup result obtained on the keratin materials. The finishing is performed, for example, by rubbing the surface to be made up with the device 1 to obtain special effects.

FIG. 6 shows an embodiment example of a cosmetic assembly 10 according to the invention. This assembly comprises, in the same packaging, a plurality of devices 1 according to the invention which each differ by the pattern formed by and/or the colour of the coat 4. The packaging may be leaktight so as to prevent the inks from drying out. The packaging may be made with means for avoiding contact of the inks with a surface other than the transfer surface, so as to reduce the risk of premature transfer. For example, the packaging comprises a thermoformed shell whose wall extends a distance from the areas of the substrate that are covered with inks.

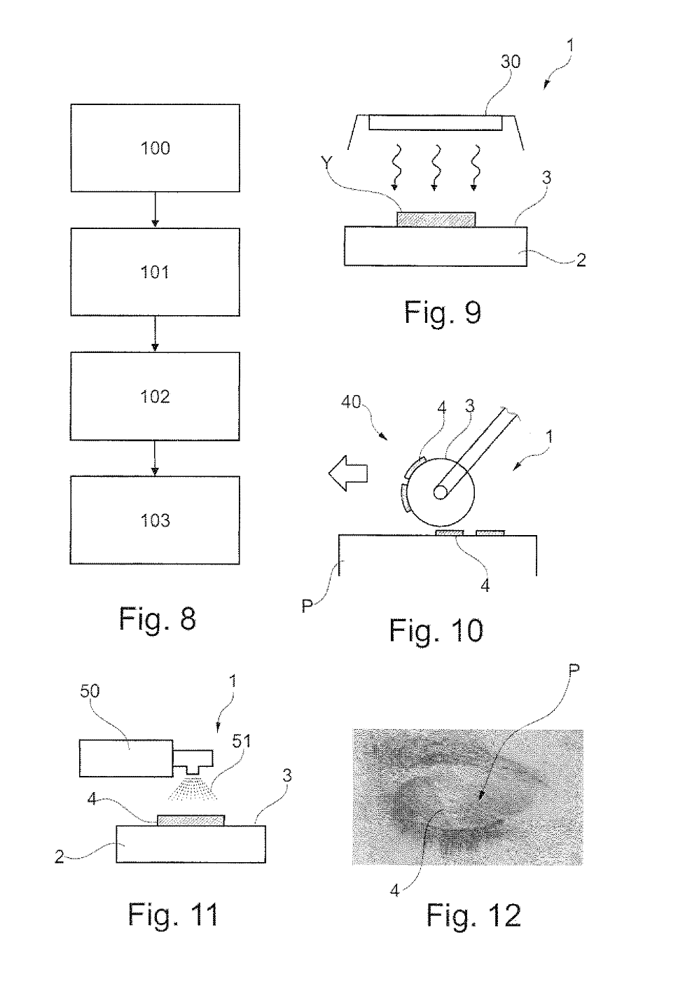

An example of a process for manufacturing a device according to the invention will now be described, with reference to FIG. 8.

In a first step 100, various patterns are proposed to the user, for example by displaying on the screen of a machine. Step 101 of choosing the pattern by the user may comprise an action such as pressing on a touchscreen in order to select the pattern intended to be printed.

The machine may also provide the user with a simulation of the makeup result. Thus, the machine may display a simulation of the appearance of the keratin materials made up with the chosen or produced pattern. To do this, the machine may acquire at least one image of the keratin materials to be made up.

In one variant, the user makes a computer file with the pattern that he wishes to print. In this case, the user may use drawing software for making such a pattern, and edit it, for example, in a file in image format.

Once the pattern has been chosen or made, the machine sends to the printer the data necessary for printing the pattern in step 102.

The machine may be connected physically and/or by means of a network to the printer performing the printing.

Once the data have been received, the pattern is printed in step 103.

The printer driver may comprise a menu for selecting a cosmetic ink cartridge among other cartridges installed in the printer and/or the nature of the substrate that is printed. As a variant, the printer automatically recognizes that the cartridge installed contains a cosmetic ink according to the invention and adjusts the operating parameters in consequence. The cartridge may thus comprise an identifier, for example an electronic chip, for providing the printer with information relating to the nature of the colouring ink that it contains, especially that this ink is of cosmetic nature.

In one embodiment example, the printer is configured to prohibit printing if the presence of a cartridge comprising a composition not intended to be placed in contact with human keratin materials, especially the skin, the nails or the lips, is detected.

As a variant, the printer may perform printing even if the presence of a cartridge comprising a composition not intended to be placed in contact with human keratin materials, especially the skin, the nails or the lips, is detected, this non-cosmetic ink cartridge possibly being used for printing on the substrate an indication relating to the cosmetic colouring ink borne by the transfer surface and/or the nature of the keratin materials to be made up.

The printing of the substrate may take place in several passes, to make successive deposits of ink at the same place, so as to increase the amount of ink deposited on the substrate. The substrate may effect, for example, between 1 and 20 passes in the printer and the amount of cosmetic ink dry matter deposited ranges, for example, from 0.01 mg/cm² to 100 mg/cm², or even from 0.1 mg/cm² to 10 mg/cm², better still from 0.2 mg/cm² to 10 mg/cm², in particular from 0.2 mg/cm² to 5 mg/cm².

The pattern may be monochromatic or, better still, polychromatic. In this case, printing may be performed at each passage in the printer with several cosmetic inks that are locally juxtaposed at the microscopic scale, depending on the colour to be reproduced.

The printing resolution may be between 16 dpi and 1600 dpi.

The printer may be designed to detect whether the ink previously deposited on the substrate is sufficiently dry before printing a new coat of ink, for example by measuring the electrical conduction between two points.

The printer and/or the printer driver may be made so as to inform the user of the need to wait a predefined time before performing a new printing on the already-printed substrate. The printer and/or the driver may automatically suspend the printing of an already-printed substrate if sufficient time has not passed to allow sufficient drying. The printer is preferably arranged so as not to deliver the printed substrate as long as all the coats of ink to be printed have not been printed.

FIG. 7 shows an embodiment example of a cosmetic assembly 20 according to the invention. The cosmetic assembly 20 comprises, in the same packaging:
a) a printer cartridge 21 comprising a cosmetic colouring ink according to the invention, and
b) a transfer surface 3 intended to be printed with the colouring ink, for example defined by a substrate sheet.

This cosmetic assembly may be provided to the user, where appropriate, with the printer intended to use the cartridge.

FIG. 9 illustrates a process variant according to the invention, in which the transfer surface 3 is exposed to the heat of a heating member 30, such that the coat of colouring ink 4 is fluidized, being, for example, initially in a solid or less fluid form.

The coat of colouring ink 4 thus thermally fluidized is then placed in contact with the keratin materials to be made up, optionally after a period of time sufficient for the coat of colouring ink to lose, for example, at least 5° C., relative to the maximum temperature obtained from the heating, but still remains sufficiently hot.

FIG. 11 shows another process variant according to the invention, in which a solvent such as water 51 is sprayed onto the coat of ink 4. The said coat is, for example, in solid form, and the solvent thus sprayed on allows it to be wetted. The ink may also not be solid, but the solvent that is sprayed on helps to make it more fluid. The colouring ink, once wetted, is then placed in contact with the keratin materials. The solvent is sprayed, for example, using a pressurized container 50 of aerosol type, actuated by the user.

FIG. 10 shows an embodiment variant of the device according to the invention in which the transfer surface 3 consists of the outer surface of an applicator roller 40, on which the coat of ink is present. Such a surface may advantageously make it possible to perform transfer makeup application onto extended areas such as the back, the tummy or the legs.

### EXAMPLES

### Example 1

Four compositions according to the invention corresponding to the formulations given in the table below were prepared:

TABLE 1

| | Yellow I | Magenta I | Cyan I | Black I |
|---|---|---|---|---|
| Dye | D&C | FD&C Red 4 | FD&C Blue 1 | (1) |
| | Yellow 8 | 1% | 1% | 1% |
| | 1% | | | |
| Ethylene glycol | | 4% | 6% | 5% |
| Diethylene glycol | 8% | | | |
| 1,5-Pentanediol | | 4% | 4% | |
| 2-Pyrrolidone | 5% | 5% | 4% | |
| Glycerol | 8% | 3% | 4% | 7% |
| 2-Imidazolidinone | 4% | 4% | 4% | 9% |
| Water | 76% | 79% | 77% | 78% |
| Total | 100% | 100% | 100% | 100% |

(1) Brown-Replacement-J from Sensient

Four compositions outside the invention corresponding to the formulations given in the table below were prepared:

TABLE 2

| | Yellow R | Magenta R | Cyan R | Black R |
|---|---|---|---|---|
| Dye | D&C | FD&C Red 4 | FD&C Blue 1 | (1) |
| | Yellow 8 | 1% | 1% | 1% |
| | 1% | | | |
| Ethanol | 6% | 6% | 6% | 5% |
| 2-Propanol | 12% | | 6% | |
| (boiling point: | | | | |
| 97° C.) | | | | |
| 1-Butanol | | 10% | 5% | |
| (boiling point: | | | | |
| 117° C.) | | | | |
| Acetone | 3% | 5% | 5% | 4% |
| Dimethyl carbonate | | | | 10% |
| (boiling point: | | | | |
| 90° C.) | | | | |
| Water | 78% | 78% | 77% | 80% |
| Total | 100% | 100% | 100% | 100% |

These compositions are placed in Canon printer cartridges, and then used with a Canon Pixma IP100 inkjet printer, which is requested to print a makeup pattern produced with the PowerPoint® software, for example. The printing is performed on a transparent plastic sheet for a commercial printer (smooth side), using each of the inks independently.

After each printing, a waiting time of 8 minutes is observed and the transfer surface is then applied to an area of skin, for example the arm.

A sharp and clearly visible pattern is obtained in the case of the formulations according to the invention. Conversely, the patterns are poorly visible or not visible in the case of the formulations outside the invention.

### Example 2

### Corresponding to FIG. 12

A makeup pattern is produced using different colours.

This pattern comprises a black line and a shaded-off area. The two parts of the pattern are separated from each other to bring out the precision of the makeup result.

As in Example 1, a transparent plastic printer sheet is used as substrate for receiving the coat of ink.

The inks according to the invention of Example 1 are printed onto the plastic sheet. The same experiment is performed using the formulations outside the invention of Example 1.

After 5 minutes, the sheets are applied to the eyelid to perform the transfer.

Only the pattern produced with the inks according to the invention allows the transfer to be achieved.

The result is precise and perceived as being inaccessible by conventional makeup means.

Within two minutes, as illustrated in FIG. 12, the pattern was spread at its top part, for the purpose of softening the edges and thus making the demarcations disappear.

Conversely, care is taken not to spread the bottom area (line and space between line and area).

### Example 3

### Example of Formulation with Water-Immiscible Transfer Compounds

Four compositions according to the invention corresponding to the formulations given in the table below were prepared:

| | Yellow I | Magenta I | Cyan I | Black I |
|---|---|---|---|---|
| Dye | D&C | FD&C Red 4 | FD&C Blue 1 | (1) |
| | Yellow 8 | 1% | 1% | 1% |
| | 1% | | | |
| Benzyl alcohol | 8% | 8% | 8% | 8% |
| Ethanol | 10% | 10% | 10% | 10% |
| Water | 81% | 81% | 81% | 81% |
| Total | 100% | 100% | 100% | 100% |

(1) Brown-Replacement-J from Sensient

The process is performed as in Example 1 using these compositions introduced into Canon printer cartridges and then used with a Canon Pixma IP100 inkjet printer.

For each ink, after printing on the smooth side of a transparent plastic sheet for a commercial printer, a waiting time of 8 minutes is observed and the transfer surface is then applied to an area of skin, for example the arm.

A sharp and clearly visible pattern is obtained for each ink in the case of the formulations according to the invention.

The expression “comprising a” should be understood as being synonymous with “comprising at least one”.

The expression “between . . . and . . . ” or “ranging from . . . to . . . ” should be understood as including the limits.

1. A transfer device for making up human keratin materials, comprising:
a substrate having at least one transfer surface, and
a coat of at least one cosmetic colouring ink borne by the transfer surface and obtained by printing using at least one digital printer, the colouring ink being intended to be applied to the keratin materials so as to obtain by transfer a makeup result on the keratin materials, the colouring ink comprising:
a dyestuff, and
a transfer compound other than the dyestuff and having a boiling point of greater than or equal to 120° C.

2. The device according to claim 1, the transfer compound(s) being chosen from glycerol, ethylene glycol, diethylene glycol, triethylene glycol, 1,5-pentanediol, 1-pentanol, 1-hexanol, benzyl alcohol, 1-hexanal, 1-heptanal, 2-pyrrolidone, N-methyl-2-pyrrolidone, N-ethylpyrrolidine, propylene carbonate, 1,3-diaminopropane, 2-imidazolidinone, 2-amino-1-butanol, 2-aminopropanol, ethanolamine; butyl acetate, and mixtures thereof.

3. The device according to claim 1, the colouring ink comprising at least one water-miscible transfer compound.

4. The device according to claim 1, the colouring ink comprising at least one water-immiscible transfer compound, preferably chosen from butyl acetate, benzyl alcohol, isododecane and isopropyl myristate, and mixtures thereof.

5. The device according to claim 1, the colouring ink also comprising a liquid solvent with a boiling point of less than or equal to 120° C. and/or the colouring ink not comprising any pigment and/or the colouring ink not comprising any particulate filler and/or the colouring in having a viscosity ranging from 1 mPa·s to 500 mPa·s, when measured at 25° C. according to a conventional process using a Rheomat 180 viscometer equipped with a spindle rotating at 200 rpm.

6. The device according to claim 1, the transfer compound(s) being fluid at a temperature of 20° C.

7. The device according to claim 1, the transfer compound(s) being present in the colouring ink in a mass content ranging from 2% to 80%, relative to the total mass of the colouring ink, and/or the transfer compound(s) having a boiling point of greater than or equal to 125° C. and/or the dyestuff being present in the colouring ink in a mass content ranging from 0.01% to 60%, relative to the total mass of the colouring ink, and the dyestuff consisting of one or more dyes and/or the colouring ink comprising water, in which the dyestuff and the transfer compound are present, the water being present in the colouring ink in a mass content ranging from 19.9% to 97.9%, relative to the total mass of the colouring ink.

8. The device according to claim 1, the transfer surface being defined by all or part of: the outer surface of an applicator roller, the surface of an applicator pad, an element in sheet form, a patch, the surface of a porous foam, a coarse brush, a fine brush or a flocked tip.

9. The device according to claim 1, the substrate comprising an indication regarding the nature of the keratin materials intended to be made up with the colouring ink and/or the substrate comprising at least one translucent or transparent area.

10. The device according to claim 1, the coat of colouring ink comprising several colouring inks of different colours, each deposited in raster dots.

11. The device according to claim 1, the colouring ink being capable of transferring onto the keratin materials without addition of an intermediary fluid compound.

12. A cosmetic assembly comprising, in the same packaging, a plurality of different devices, each being according to claim 1, the devices differing by the chemical nature of the colouring they bear and/or by the pattern thereby formed and/or by the form of the transfer surface intended to engage with the keratin materials.

13. A process for manufacturing a device according to claim 1, comprising the step that consists in printing, using at least one digital printer, on a transfer surface a coat of at least one cosmetic colouring ink, the colouring ink comprising:
a dyestuff, and
a transfer compound other than the dyestuff, having a boiling point of greater than or equal to 120° C.,
the colouring ink being printed in a predefined pattern.

14. A cosmetic assembly for manufacturing a device according to claim 1, comprising, in the same packaging:
a) a printer cartridge comprising a cosmetic colouring ink, the colouring ink comprising:
a dyestuff,
a transfer compound other than the dyestuff and having a boiling point of greater than or equal to 120° C., and
b) a transfer surface intended to be printed with the colouring ink.

15. A process for making up human keratin materials, comprising the step consisting in applying to the keratin materials the colouring ink(s) present on a device according to claim 1.

16. The process according to claim 15, the keratin materials intended to be coated with the colouring ink(s) not having been covered, before application of the colouring ink(s), with an intermediary fluid compound, intended to improve the transfer of the colouring ink(s) and/or the process lacking a step of addition to the colouring ink(s) borne by the transfer surface of an intermediary fluid compound intended to improve the transfer.
